(19) United States
(12) Patent Application Publication
(10) Pub. No.: US 2016/0091359 A1
(43) Pub. Date: Mar. 31, 2016

(54) SYSTEM AND METHOD FOR MEASURING SEGMENT PARAMETERS OF A HUMAN BODY FOR RECORDING A BODY MASS INDEX

(71) Applicant: Cladoop, INC, Murphy, TX (US)

(72) Inventors: Maqsood Alam, Murphy, TX (US); Muzammil Alam, Hyderabad (IN)

(21) Appl. No.: 14/501,072

(22) Filed: Sep. 30, 2014

(51) Int. Cl. G01G 19/50; A61B 5/117; A61B 5/107; A61B 5/00

(52) U.S. Cl. CPC G01G 19/50; A61B 5/0077; A61B 5/1176; A61B 5/1072; A61B 5/1079; A61B 5/4872; A61B 5/7278; A61B 5/0013; A61B 2560/0475

(57) ABSTRACT

Exemplary embodiments of the present disclosure are directed towards a system and method for measuring segment parameters of a human body for recording a body mass index. The method includes a digital weighing scale configured to measure a weight of specimen and the measured weight of the specimen instantly transmitted to a server along with an identical time, an image capturing unit positioned in a predetermined distance away from the digital weighing scale at a preferred location for capturing an image of the specimen, a face recognition unit triggered by the digital weighing scale for comparing the image captured by the image capturing unit with the predetermined data provided by the specimen, a height strip placed vertically on the digital weighing scale configured to measure a height of the specimen, a body mass index calculating unit configured to calculate a body mass index of the specimen with the weight and the height measured by the digital weighing scale and the height strip and a data repository unit configured to store dynamically the measured data of the specimen.

### TECHNICAL FIELD

The present disclosure relates to a body mass index measuring system. More particularly, the present disclosure relates to a system and method for measuring segment parameters of a human body for recording a body mass index.

### BACKGROUND

Typically, a conventional digital weighing scale includes a digital data processing apparatus for computing and displaying a quantity representative of or proportional to weight on the digital weighing scale in accordance with a computational algorithm which is at least partly determined by variable parameters selected by a user of the digital weighing scale. However, there is a problem that the conventional digital weighing scale cannot transmit the measured data through an Internet network and also cannot provide body mass index of the user.

The existing body measuring instrument including the scale body, a display unit and input keys of the load detecting circuit. The Body Mass Index is generally obtained by dividing the person's bodyweight by the square of person's height. For measuring the body mass index it is required to measure the height and weight separately, this method consumes the time for providing the body mass index of the person.

In the light of aforementioned discussion there exists a need of a system and method for measuring segment parameters of a human body for recording a body mass index.

### BRIEF SUMMARY

The following presents a simplified summary of the disclosure in order to provide a basic understanding to the reader. This summary is not an extensive overview of the disclosure and it does not identify key/critical elements of the invention or delineate the scope of the invention. Its sole purpose is to present some concepts disclosed herein in a simplified form as a prelude to the more detailed description that is presented later.

A more complete appreciation of the present disclosure and the scope thereof can be obtained from the accompanying drawings which are briefly summarized below and the following detailed description of the presently preferred embodiments.

Exemplary embodiments of the present disclosure are directed towards a system and method for measuring segment parameters of a human body for recording a body mass index.

According to a first aspect, the system for measuring segment parameters of a human body includes a digital weighing scale configured to measure a weight of specimen. The measured weight of the specimen instantly transmitted to a server along with an identical time. The digital weighing scale transmits a trigger to the image capturing unit for capturing the image of the specimen. The server configured to receive the measured data of an individual specimen from the digital weighing scale.

According to the first aspect, the system includes an image capturing unit positioned at a predetermined distance away from the digital weighing scale and at a preferred location for capturing an image of the specimen.

According to the first aspect, the system includes a face recognition unit triggered by the digital weighing scale for comparing the image captured by the image capturing unit with the predetermined data provided by the specimen. The measured data of the specimen automatically transmitted to the body mass index calculating unit for calculating the body mass index corresponding to the specimen.

According to the first aspect, the system includes a height strip placed vertically or placed at the back of the weighing scale and within focus of camera and configured to measure a height of the specimen. The measured height of the specimen read by the face recognition unit from the height strip and captured image of the specimen.

According to the first aspect, the system includes a body mass index calculating unit conFlGd to calculate a body mass index of the specimen with the weight and the height measured by the digital weighing scale and the height strip.

According to the first aspect, the system includes a data repository unit configured to dynamically store the measured data of the specimen. The stored data corresponding to each individual specimen arranged in a preferred manner in at least one of weight, height and body mass index of the corresponding specimen.

According to the first aspect, the system includes a communication interface configured to receive a current measured value of the specimen from the digital weighing scale and transmit the measured weight to the server.

According to a second aspect, the method includes measuring a weight of specimen by a digital weighing scale. The measured weight of the specimen instantly transmitted to a server along with an identical time.

According to a second aspect, the method includes capturing an image of the specimen by an image capturing unit positioned in a predetermined distance away from the digital weighing scale at a preferred location.

According to a second aspect, the method includes triggering a face recognition unit by the digital weighing scale for comparing the image captured by the image capturing unit with the predetermined data provided by the specimen.

According to a second aspect, the method includes measuring a height of the specimen by a height strip placed vertically or placed at the back of the weighing scale and within focus of camera configured to measure a height of the specimen. The measured height of the specimen read by the face recognition unit from the height strip and captured image of the specimen. The method further includes measuring the height of the specimen by the height strip hidden by the specimen's body.

According to a second aspect, the method includes calculating a body mass index of the specimen with the weight and the height measured by the digital weighing scale and height strip by a body mass calculating unit.

According to a second aspect, the method includes dynamically storing the measured data of the specimen in a data repository unit. The stored data corresponding to each individual specimen arranged in a preferred manner in at least one of weight, height and body mass index of the corresponding specimen.

### BRIEF DESCRIPTION OF DRAWINGS

Other objects and advantages of the present invention will become apparent to those skilled in the art upon reading the following detailed description of the preferred embodiments, in conjunction with the accompanying drawings, wherein like reference numerals have been used to designate like elements, and wherein:

FIG. 1 is a diagram depicting a system for measuring segment parameters of a human body to record a body mass index, according to an exemplary embodiment of the present disclosure.

FIG. 2 is an exemplary block diagram describing an automated digital weighing scale for recording a body mass index of the specimen, according to an exemplary embodiment of the present disclosure.

FIG. 3 is a flow diagram depicting a method for measuring segment parameters of a human body to record a body mass index, according to an exemplary embodiment of the present disclosure.

### DETAILED DESCRIPTION

It is to be understood that the present disclosure is not limited in its application to the details of construction and the arrangement of components set forth in the following description or illustrated in the drawings. The present disclosure is capable of other embodiments and of being practiced or of being carried out in various ways. Also, it is to be understood that the phraseology and terminology used herein is for the purpose of description and should not be regarded as limiting.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced item. Further, the use of terms “first”, “second”, and “third”, and the like, herein do not denote any order, quantity, or importance, but rather are used to distinguish one element from another.

FIG. 1 is a diagram 100 illustrating a diagram depicting a system for measuring segment parameters of a human body to record a body mass index. According to a non limiting exemplary embodiment of the present disclosure, the system includes a digital weighing scale 102 which is used to measure a weight of a specimen (also referred as human body) along with an identical time. The digital weighing scale 102 communicates with the server through a data communication network, thereby the digital weighing scale 102 transmitting the measured data to the server. A height strip 104 is placed vertically on the digital weighing scale 102 for measuring the height of the specimen. The digital weighing scale 102 transmits a trigger to an image capturing unit 106 which is positioned in a predetermined distance away from the digital weighing scale 102 at a preferred location and for capturing an image of the specimen. Though not depicted in FIG. 1, a face recognition unit receives the captured image of the specimen for comparing the received image with the predetermined data provided by the specimen. A body mass index calculating unit receives the measured height, weight from the digital weighing scale 102, the height strip and the capturing unit 106 for calculating the body mass index of the specimen. Then the calculated data is transmitted to a data repository unit for storing dynamically in a predetermined manner.

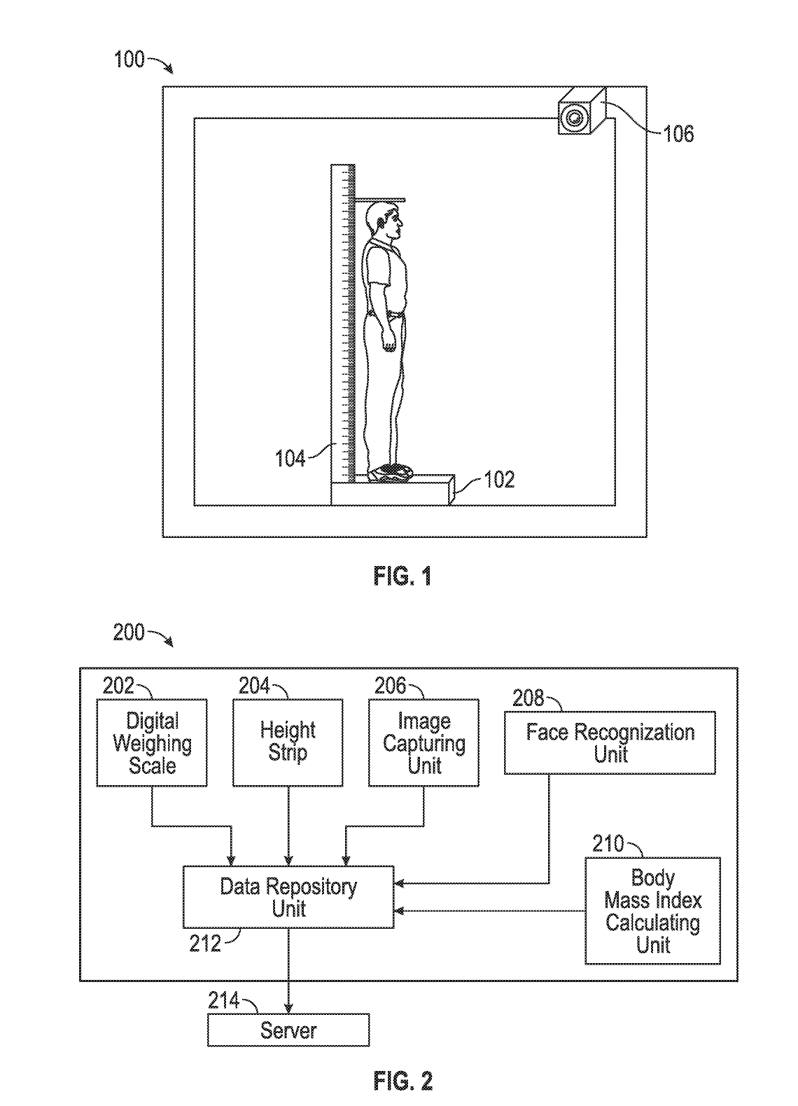

FIG. 2 is a diagram 200 illustrating an exemplary block diagram describing an automated digital weighing scale for recording a body mass index of the specimen. According to a non limiting exemplary embodiment of the present disclosure, the diagram includes a digital weighing scale 202 possessing a weighing pad to measure the weight of a specimen and the measured weight of the specimen and to instantly transmit to a server along with an identical time. A height strip 204 is arranged vertically on the digital weighing scale 202 for detecting the height of the specimen which is measures the specimen with varying heights (3 to 7 FT). Simultaneously, the digital weighing scale 202 transmits a trigger to an image capturing unit 206 for capturing a face of the specimen. The image capturing unit 206 is positioned in a predetermined distance away from the digital weighing scale 202 at a preferred location then the captured image is transmitted to a face recognition unit 208 for comparing the image captured by the image capturing unit 106 with the predetermined data provided by the specimen.

As shown in FIG. 2, a body mass index calculating unit 210 is used to receive the measured height and weight of the specimen from the digital weighing scale 202 and the height strip 204 for calculating a body mass index of the specimen. Further the measured data is dynamically stored in a data repository unit 212 for storing the data corresponding to each individual specimen in a preferred manner as weight, height and body mass index of the corresponding specimen. The data collected by the digital weighing scale 202, the height strip 204 and face recognition unit 208 are stored in a server 214 through a data communication network and the data is stored in the data repository unit 212 to further update in the server 214.

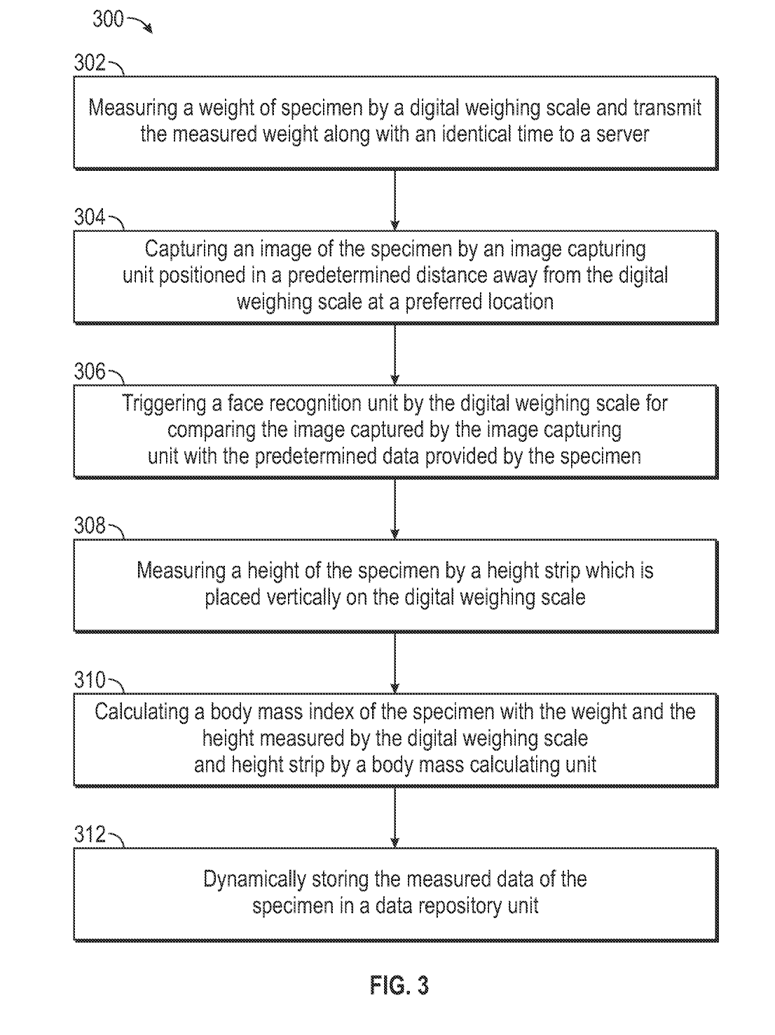

FIG. 3 is a diagram 300 illustrating a flow diagram of a method for measuring segment parameters of a human body to record a body mass index. The method starts at a step 302, wherein a specimen weight measured by a digital weighing scale and instantly transmitted to a server along with an identical time. At step 304, the image capturing unit captures a face of the specimen which is placed at a predetermined distance away from the digital weighing scale. Then the weighing scale transmits a trigger to the face recognition unit for comparing the image captured by the image capturing unit with the predetermined data provided by the specimen at step 306. Next at step 308, the height of the specimen measured by a height strip which is placed vertically on the digital weighing scale and measured height read by the face recognition unit. Further at step 310, a body mass index of the specimen calculates by a body mass calculating unit which is based on the weight and the height measured by the digital weighing scale, the height strip and the captured image of the capturing unit. Finally at step 312, a data repository unit is used to dynamically store the measured data of the specimen corresponding to each individual specimen arranged in a preferred manner.

While specific embodiments of the disclosure have been shown and described in detail to illustrate the inventive principles, it will be understood that the disclosure may be embodied otherwise without departing from such principles.

What is claimed is:

1. A system for measuring segment parameters of a human body to record a body mass index , comprising:
a digital weighing scale configured to measure a weight of specimen, whereby the measured weight of the specimen instantly transmitted to a server along with an identical time;
an image capturing unit positioned in a predetermined distance away from the digital weighing scale at a preferred location for capturing an image of the specimen;
a face recognition unit triggered by the digital weighing scale for comparing the image captured by the image capturing unit with the predetermined data provided by the specimen;
a height strip placed vertically on the digital weighing scale configured to measure a height of the specimen, whereby the measured height of the specimen read by the face recognition unit from the height strip and captured image of the specimen;
a body mass index calculating unit configured to calculate a body mass index of the specimen with the weight; and the height measured by the digital weighing scale; and the height strip; and
a data repository unit configured to dynamically store the measured data of the specimen, whereby the stored data corresponding to each individual specimen arranged in a preferred manner in at least one of: weight; height; and body mass index of the corresponding specimen.

2. The system of claim 1, wherein the digital weighing scale transmits a trigger to the image capturing unit for capturing the image of the specimen.

3. The system of claim 1, wherein the server configured store to receive the measured data of the individual specimen from the digital weighing scale.

4. The system of claim 1, wherein the measured data of the specimen automatically transmitted to the body mass index calculating unit for calculating the body mass index corresponding to the specimen.

5. The system of claim 1, wherein a communication interface configured to receive a current measured value of the specimen from the digital weighing scale and transmit the measured weight to the server.

6. The system of claim 1, wherein the height strip placed at the back of the weighing scale and within the focus of the image capturing unit for measuring the height of the specimen.

7. A method for measuring segment parameters of a human body to record a body mass index , the method comprising:
measuring a weight of specimen by a digital weighing scale, whereby the measured weight of the specimen instantly transmitted to a server along with an identical time;
capturing an image of the specimen by an image capturing unit positioned in a predetermined distance away from the digital weighing scale at a preferred location;
triggering a face recognition unit by the digital weighing scale for comparing the image captured by the image capturing unit with the predetermined data provided by the specimen;
measuring a height of the specimen by a height strip placed vertically on the digital weighing scale, whereby the measured height of the specimen read by the face recognition unit from the height strip and captured image of the specimen; and
calculating a body mass index of the specimen with the weight; and the height measured by the digital weighing scale; and height strip by a body mass calculating unit; and
dynamically storing the measured data of the specimen in a data repository unit, whereby the stored data corresponding to each individual specimen arranged in a preferred manner in at least one of: weight; height; and body mass index of the corresponding specimen.

8. The method of claim 7, further comprising a step of measuring the height of the specimen by the height strip hidden by the specimen's body.
